(19) United States
(12) Patent Application Publication
(10) Pub. No.: US 2024/0416062 A1
(43) Pub. Date: Dec. 19, 2024

(54) MASK WITH INTEGRATED CAPNOGRAPHY PORT

(71) Applicant: Parker-Hannifin Corporation, Cleveland, OH (US)

(72) Inventors: David J. ROWE, Parkesburg, PA (US); Seth B. WOLF, North Wales, PA (US); Yuhsuan CHEN, Somerville, NJ (US); Stephen E. SIMANTIRAS, Randolph, NJ (US); Robert D. BOYER, Boonton, NJ (US); Justin T. NIEMEIER, Bloomingdale, NJ (US); Hannah M. TESON, Sinking Spring, PA (US); Michael V. QUINN, East Hanover, NJ (US)

(73) Assignee: Parker-Hannifin Corporation, Cleveland, OH (US)

(21) Appl. No.: 18/705,097

(22) PCT Filed: Nov. 18, 2022
(86) PCT No.: PCT/US2022/080099

§ 371 Date: Apr. 26, 2024

Related U.S. Application Data
(60) Provisional application No. 63/308,558, filed on Feb. 10, 2022.

(51) Int. Cl. A61M 16/06; A61M 16/08

(52) U.S. Cl. CPC A61M 16/06; A61M 16/085; A61M 16/0875; A61M 16/0891; A61M 2205/33; A61M 2210/0618; A61M 2230/43

(57) ABSTRACT

A capnography mask includes a mask body having a cover portion configured to cover at least the nostrils of a patient's nose and forms a breathing cavity for inhalation of inflow gas and exhalation of expired gas. The mask also includes an inflow port fluidly connected to the breathing cavity for delivering the inflow gas to the breathing cavity, an outflow port fluidly connected to the breathing cavity for delivering the expired gas out of the breathing cavity, and a capnography port fluidly connected to the breathing cavity for delivering a sample of the expired gas to a capnography machine. The inflow, outflow and capnography ports may be unitarily formed by respective portions of the mask body to form a unitary mask structure. The ports may include respective tube segments that may cooperate with respective ribs to distribute load away from the alar fibrofatty tissue of the patient's nose, thereby improving comfort.

### RELATED APPLICATIONS

This application claims the benefit of U.S. Provisional Application No. 63/308,558 filed Feb. 10, 2022, which is incorporated herein by reference in its entirety.

### TECHNICAL FIELD

The present disclosure relates generally to capnography masks, and more particularly to capnography mask having an integrated capnography port.

### BACKGROUND

Capnography involves monitoring the concentration or partial pressure of carbon dioxide (CO₂) in the respiratory gases of a patient. A main development in the field of capnography has been as a monitoring tool used during anesthesia and intensive care of medical patients. Generally, there are two types of capnography. The first type is mainstream capnography which uses an in-line infrared CO₂ sensor connected directly to the airway between the breathing apparatus and the breathing circuit. The second type is sidestream capnography which pulls a sample of the patient's exhaled gas from a capnography port in the breathing circuit that is connected through tubing to an infrared sensor located in a remote monitor. Capnography results generally display a graph of expiratory CO₂ plotted against time, or the expired volume of CO₂, which serves as a visual indication that the patient is breathing properly during the medical procedure.

### SUMMARY

At least one problem with conventional sidestream capnography systems is inadequate sampling of the respiratory gas for monitoring. For example, many conventional sidestream capnography systems have a capnography connection in the breathing circuit that is complex and/or not positioned well enough to obtain an accurate sample for reading on the capnography monitor. As an example, some conventional capnography systems have the capnography port approximately three feet away from the mask at the other end of a vacuum tube connected to the mask. Due to such a long distance of the capnography port from the mask, the peaks and valleys of the capnography display graph may be diluted and flattened out, making the graph more difficult to read and monitor the patient's breathing. Other conventional sidestream capnography systems may locate the capnography port in the breathing circuit closer to the mask, but with a more complicated connection that requires additional parts and more time-consuming assembly.

At least one aspect of the present disclosure solves one or more problems of conventional capnography systems by providing a unique capnography mask having the capnography port integrated into and unitarily formed by a portion of the mask body. This locates the capnography port closer to the exhalation source of the patient, thereby improving capnography sensing and monitoring. Such a mask with the capnography port formed by the mask body also reduces the number of parts and minimizes the cost and time for assembly.

According to an aspect, a capnography mask includes: a mask body having a cover portion that is configured to cover at least the nostrils of a patient's nose and which forms a breathing cavity for inhalation of inflow gas through the patient's nose and exhalation of expired gas through the patient's nose, the mask body further comprising: an inflow port unitarily formed by a first portion of the mask body, the inflow port being fluidly connected to the breathing cavity for delivering the inflow gas to the breathing cavity; an outflow port unitarily formed by a second portion of the mask body, the outflow port being fluidly connected to the breathing cavity for delivering the expired gas out of the breathing cavity; and a capnography port unitarily formed by a third portion of the mask body, the capnography port being fluidly connected to the breathing cavity for delivering a sample of the expired gas to a capnography machine.

At least some other problems with conventional capnography masks include a lack of comfort and sealability against the patient's face. For example, some conventional masks are constructed such that load from the mask applies pressure on the alar fibrofatty tissue of the nose which can cause discomfort because this pressure can pinch and reduce the airway openings through the nostrils of the nose. In addition, some conventional masks may inadequately distribute load from the mask to the patient's face, thereby requiring the use of adhesives to form an adequate seal. While such adhesives can provide a good seal, they may be inconsistent in their ability to adhere to the patient's skin. This inconsistency can be attributed to a variety of reasons, such as shipping conditions in high humidity areas, attempting to reposition the mask on the patients face, or potentially from the natural oils on a patient's face. The adhesive also adds significant cost to the production of the mask.

At least one aspect of the present disclosure solves one or more problems with conventional capnography masks by providing a structural arrangement that distributes load to improve comfort to the patient and/or improves sealing against the patient's face. For example, the exemplary capnography mask may include an arrangement of external ribs and/or external gas ports that are oriented in such a way that load from the mask is distributed away from the alar fibrofatty tissue of the patient's nose to region(s) of the patient's face above the alar fibrofatty tissue, such as the upper boney part(s) of the patient's face and/or nose. Such load distribution reduces pinching of the patient's nostrils and improves comfort to the patient. Such a distribution of load also may improve sealability around a peripheral sealing edge of the mask against the patient's face, thereby reducing or eliminating the need for adhesives.

According to an aspect, a capnography mask includes: a mask body having a cover portion that is configured to cover at least the nostrils of a patient's nose and which forms a breathing cavity for inhalation of inflow gas through the patient's nose and exhalation of expired gas through the patient's nose; an inflow port formed by a first tube segment that extends outwardly of the cover portion on a first side of the cover portion, the inflow port being fluidly connected to the breathing cavity for delivering the inflow gas to the breathing cavity; and an outflow port formed by a second tube segment that extends outwardly of the cover portion on a second side of the cover portion that is opposite the first side, the outflow port being fluidly connected to the breathing cavity for delivering the expired gas out of the breathing cavity; wherein the first tube segment of the inflow port and the second tube segment of the outflow port each extends at an upward angle such that load from the mask is distributed away from the alar fibrofatty tissue of the patient's nose to a region of the patient's face and/or nose that is above the alar fibrofatty tissue when in use.

According to an aspect, a capnography mask includes: a mask body having a cover portion that is configured to cover at least the nostrils of a patient's nose and which forms a breathing cavity for inhalation of inflow gas through the patient's nose and exhalation of expired gas through the patient's nose; an inflow port fluidly connected to the breathing cavity for delivering the inflow gas to the breathing cavity; an outflow port fluidly connected to the breathing cavity for delivering the expired gas out of the breathing cavity; and a rib arrangement configured to distribute load away from the alar fibrofatty tissue of the patient's nose to a region of the patient's face and/or nose that is above the alar fibrofatty tissue when in use.

The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention.

FIG. 1 is a schematic view of a capnography system including an exemplary capnography mask arranged on a patient's face.

FIG. 2 is a top, front, right perspective view of the mask.

FIG. 3 is a bottom, front, left perspective view of the mask.

FIG. 4 is top, left, rear perspective view of the mask.

FIG. 5 is a top, rear, right perspective view of the mask.

FIG. 6 is a front view of the mask.

FIG. 7 is a rear view of the mask.

FIG. 8 is a top view of the mask.

FIG. 9 is a bottom view of the mask.

FIG. 10 is a right side view of the mask.

FIG. 11 is a left side view of the mask.

FIG. 12 is a front cross-sectional view of the mask taken about the line 12-12 in FIG. 10.

FIG. 13 is a cross-sectional view of the mask taken about line 13-13 in FIG. 11.

FIG. 14 is an enlarged view from the region shown in FIG. 5.

FIG. 15 is a side view of the mask and a capnography connector.

FIG. 16 is a perspective view of the mask arranged on a patient's face.

FIG. 17 is a front view of the mask arranged on the patient's face.

FIG. 18 is an enlarged view taken from the region shown in FIG. 17, in which the mask is shown in transparent view.

FIG. 19 is a side view of the mask on the patient's face in which the mask is shown in transparent view.

FIGS. 20A-20B are side views of the mask in different sizes.

FIG. 21 is a front view of another exemplary mask with a mouth portion arranged on the patient's face.

FIG. 22 is a top, left, front perspective view of another exemplary mask according to another embodiment.

FIG. 23 is a top, right, rear perspective view of the mask in FIG. 22.

FIG. 24 is a left side view of the mask in FIG. 22.

FIG. 25 is a right side view of the mask in FIG. 22.

FIG. 26 is a front cross-sectional view of the mask in FIG. 22.

### DETAILED DESCRIPTION

The principles and aspects according to the present disclosure have particular application to capnography masks such as for use in analgesia medical procedures, for example with the delivery of nitrous oxide to a patient, and thus will be described below chiefly in this context. It is understood, however, that the principles and aspects according to the present disclosure may be used for other types of capnography masks for other intensive care procedures, such as for use with anesthesia or the like.

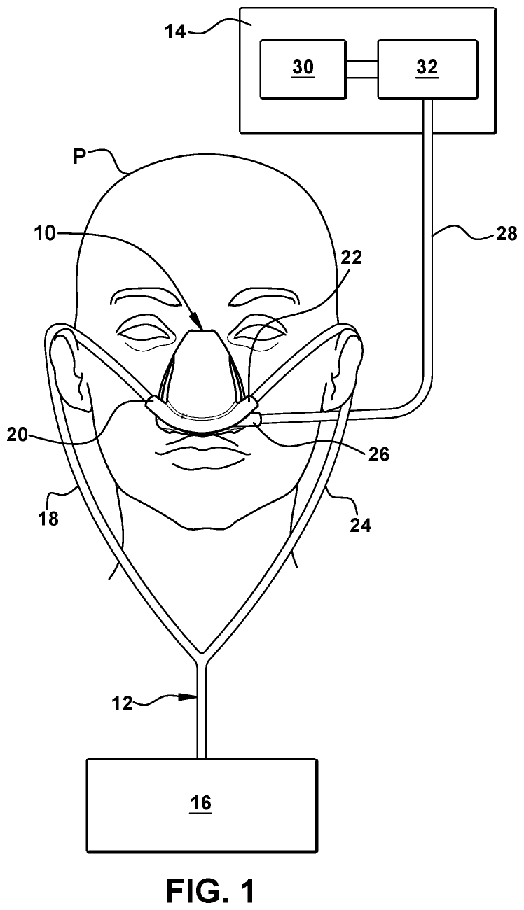

Referring to FIG. 1, an exemplary capnography mask 10 is shown arranged on the face of a patient P. The capnography mask 10 is connected in a breathing circuit 12 and to a capnography monitoring machine 14. The breathing circuit 12 includes a gas machine 16, which may include a suitable a gas supply and gas regulator, and also may include a vacuum supply for return flow. The gas machine 16 may be part of an anesthesia machine that supplies a mixture of nitrous oxide and oxygen to the patient P via gas inflow (supply) tubing 18 connected to an inflow port 20 of the mask 10. Expired gases from the patient P are fed downstream in the breathing circuit 12 via a gas outflow port 22 connected to outflow (exhalation) tubing 24 that may be routed back to the gas machine 16, an external vacuum separate from the gas machine 16, or to the external environment. The outflow tubing 24 may be connected to a regulated vacuum system (not shown) for the collection of expired gases from the mask 10 and for scavenging any anesthesia gases from the work area adjacent the mask 10.

The capnography mask 10 also includes a capnography port 26 that is connected via sampling (capnography) tubing 28 to the capnography machine 14. The capnography machine 14 may include a vacuum source 30, such as a diaphragm pump (sampling pump), that scavenges or pulls a sample of the patient's expired air/gas through the capnography port 26 and tubing 28 to a CO₂ sensor 32 located in the remotely located capnography monitoring machine 14. The CO₂ sensor 32 may be any suitable sensor, such as an infrared detector, that is adapted to detect the concentration or partial pressure of carbon dioxide (CO₂) in the respiratory gases of the patient P. The capnography machine 14 also may include a monitor for displaying the Other electromechanical equipment such as pressure transducers, solenoid valves, or the like also may be included in the capnography monitoring machine 14. The gas tubing 18, 24, 28 may be made with commercially-available flexible tubing, such as medical-grade PVC, silicone, or any other suitable material. The size of the tubing may be approximately one to five millimeters internal diameter and may use suitable connectors (not shown) for connecting the tubing 18, 24, 28 to the respective ports 20, 22, 26 of the mask 10.

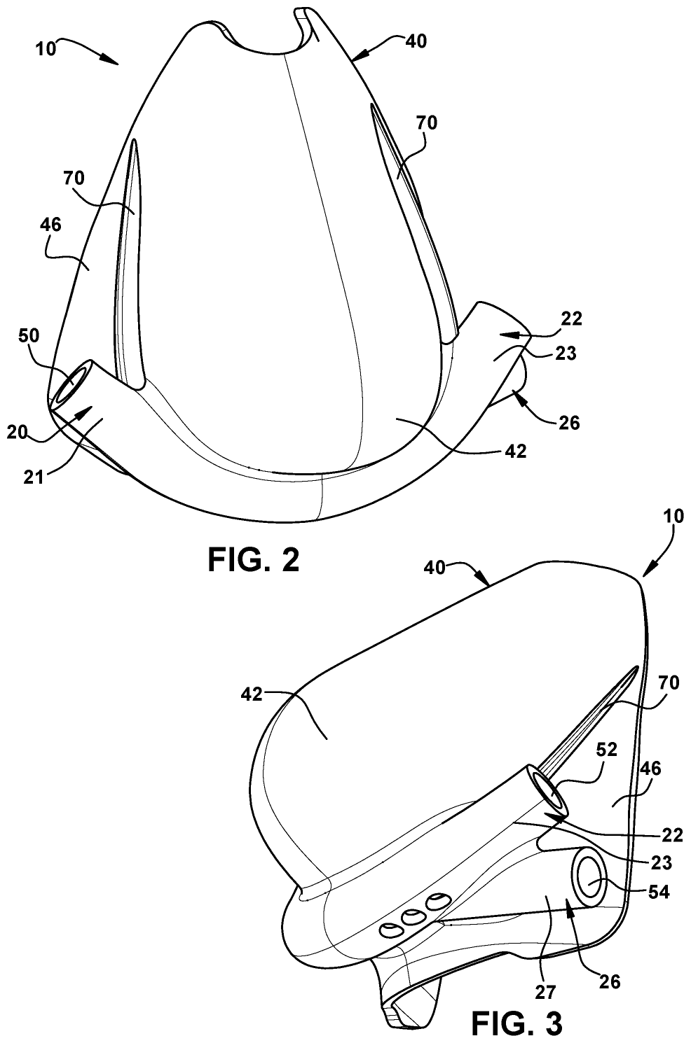

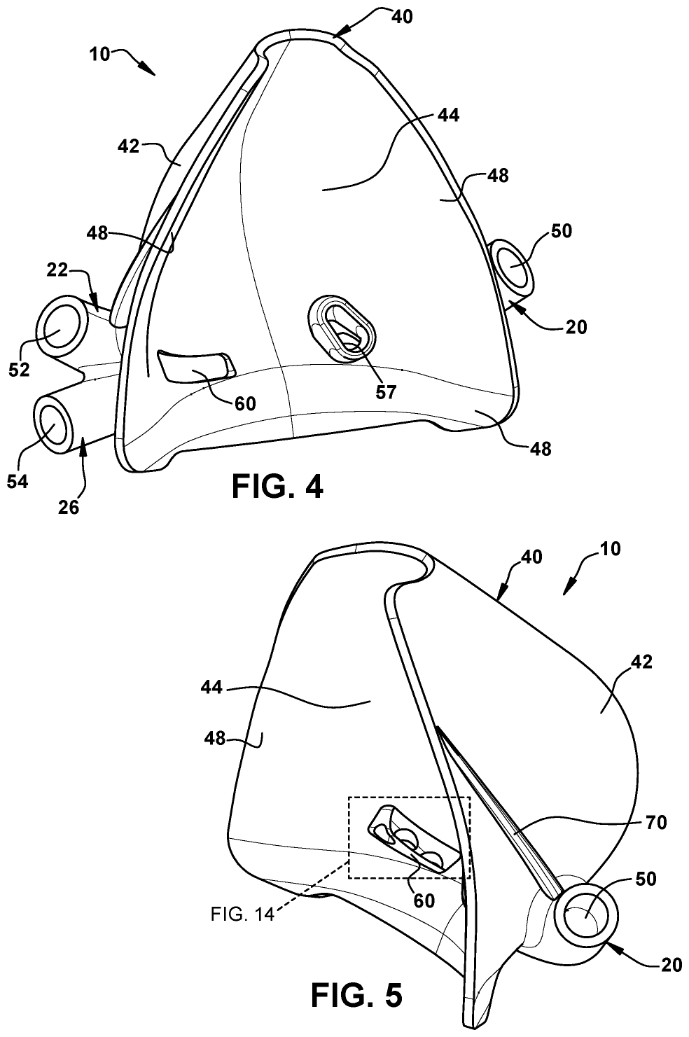

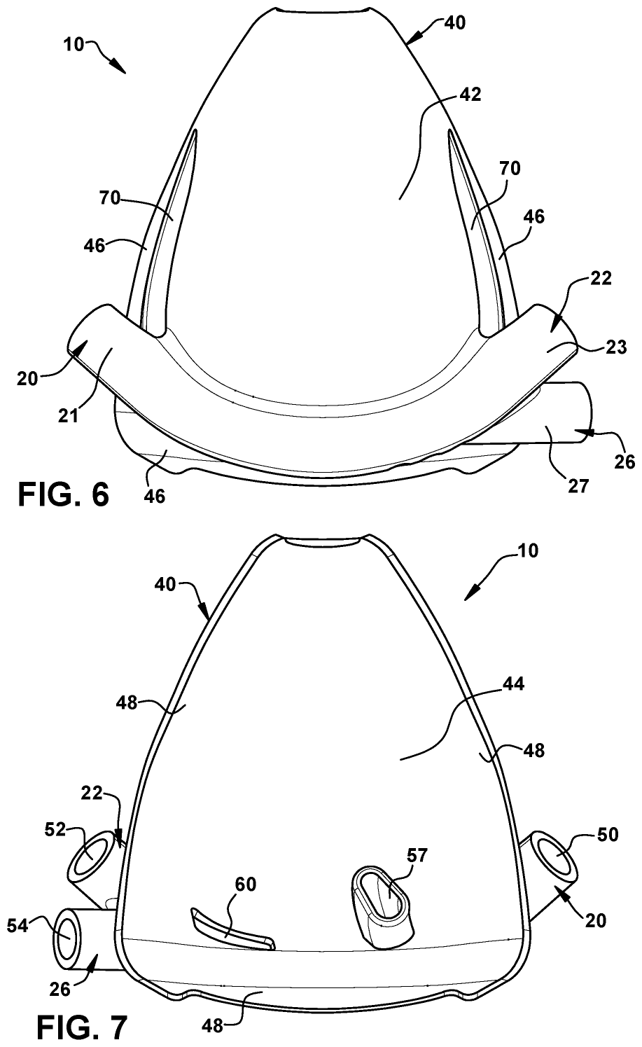

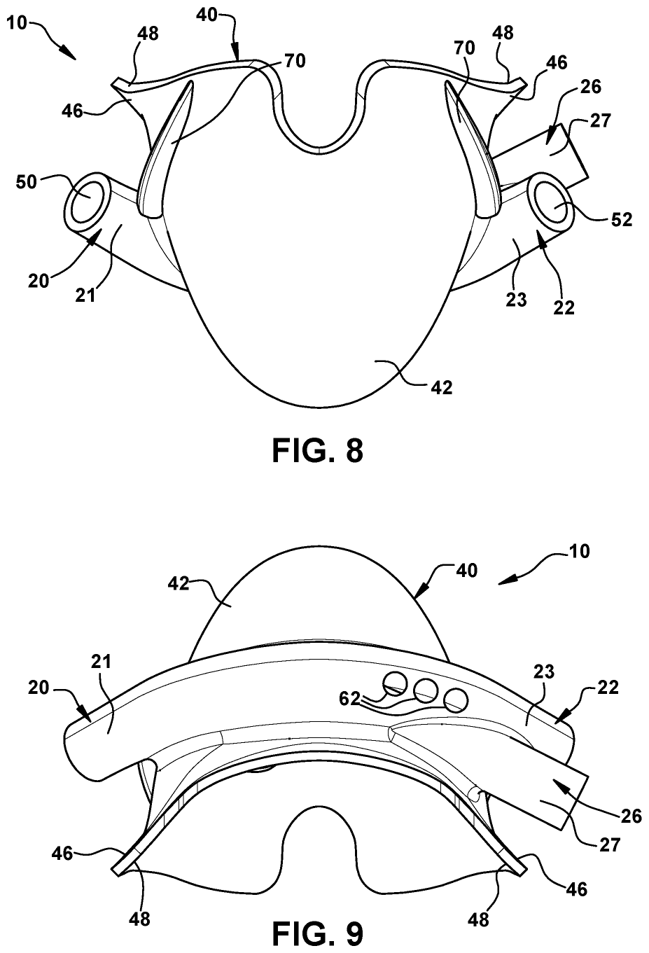

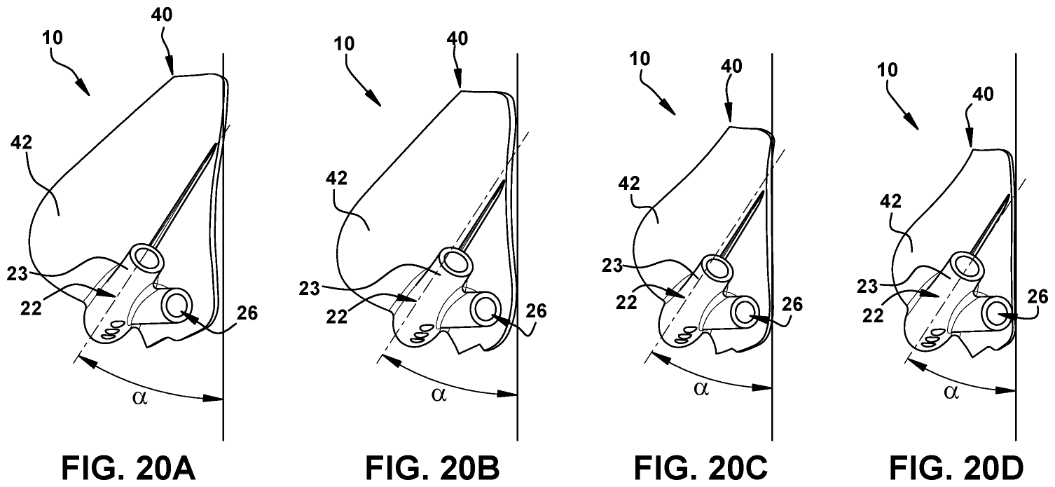

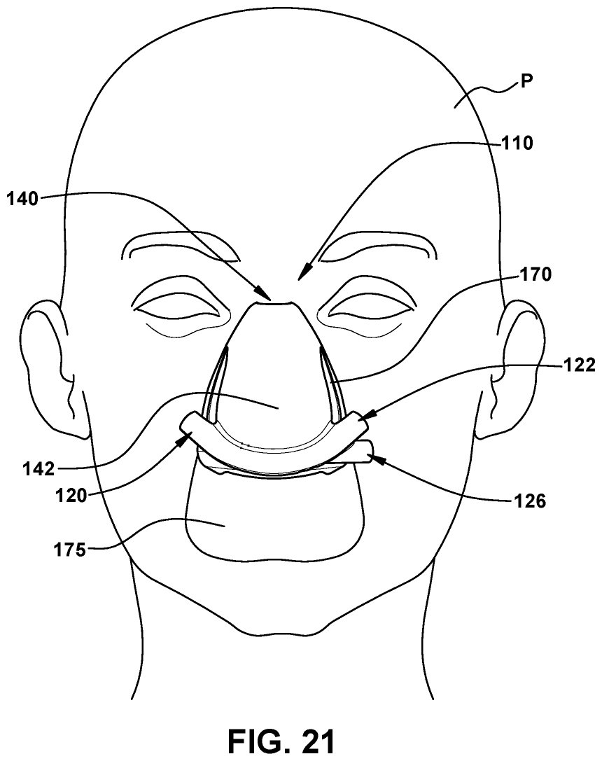

Turning to FIGS. 2-14, the exemplary capnography mask 10 is shown in further detail. As shown, the capnography mask 10 includes a mask body 40 having a cover portion 42 adapted to form a breathing cavity 44 that covers at least the nostrils of the patient's nose. In the illustrated embodiment, the mask body 40 is adapted to cover a majority of the patient's nose, including a majority of the bridge, and also may be adapted to cover the patient's mouth (as shown in FIG. 21, for example). Different sizes of masks 10 may be provided for different sized noses, which may be grouped into classifications such as adult large, adult medium, adult small, and child or pediatric size (as shown in FIGS. 20A-20D, for example).

The mask body 40 may be made with a flexible, resilient material that is molded into the nose-conforming shell. Such a flexible, resilient material may be a suitable plastic, for example a medical-grade polyvinylchloride (PVC), thermoplastic elastomer (TPE), silicone, or the like. As shown, a peripheral portion 46 of the mask may flare outwardly to form a sealing edge 48 that is adapted to substantially seal against the patient's face for containing gas within the breathing cavity 44. The flexibility of the mask 10 facilitates conformance to the patient's face and enhances the sealability of the mask at the sealing edge 48. As discussed in further detail below, the structural arrangement of the mask 10 may be configured to distribute load from the mask in a way that improves comfort to the patient and/or improves sealing against the patient's face. As such, the mask 10 may be used without adhesives at the sealing edge 48, however, it is also understood that such adhesives may be used at the sealing edge 48 as may be desired for particular applications.

As shown in the illustrated embodiment, the mask body 40 is formed as a single unitary structure, including the inflow port 20 formed by a first portion of the mask body, the outflow port 22 formed by a second portion of the mask body, and the capnography port 26 formed by a third portion of the mask body. Such a unitary mask body 40 may be made by any suitable process, such as injection molding, additive manufacturing, or the like. At least one unique attribute of the exemplary capnography mask 10 having the integrated ports 20, 22, 26 is that it reduces the number of parts, and therefore minimizes cost and time for assembling tubing to the mask 10. In addition, the unique configuration of the mask 10 with the capnography port 26 integrated into and formed by a portion of the mask body 40 provides further advantages, such as locating the capnography port closer exhalation source of the patient, thereby improving capnography sensing and monitoring, and also improving the accurate repeatability of such readings.

The inflow 20, outflow 22, and capnography ports 26 of the mask may have any suitable configuration for providing connection to the respective tubing 18, 24, 28 (shown in FIG. 1). In the illustrated embodiment, for example, each port 20, 22, 26 includes a respective tube segment 21, 23, 27 that extends outwardly of the cover portion 42 of the mask body 40. As shown, each tube segment 21, 23, 27 includes an external opening 50, 52, 54 at an end portion thereof for connecting to the respective tubing 18, 24, 28 (FIG. 1). The connection may be made by inserting the tubing into the respective opening 50, 52, 54, or may be facilitated by a connector, such as a fitting that can be inserted into the respective external opening 50, 52, 54. Because the tube segments 21, 23, 27 are a unitary part of the mask body 40 and are made from the same material, the tube segments 21, 23, 27 also may be resiliently flexible to facilitate connection of the tubing 18, 24, 28.

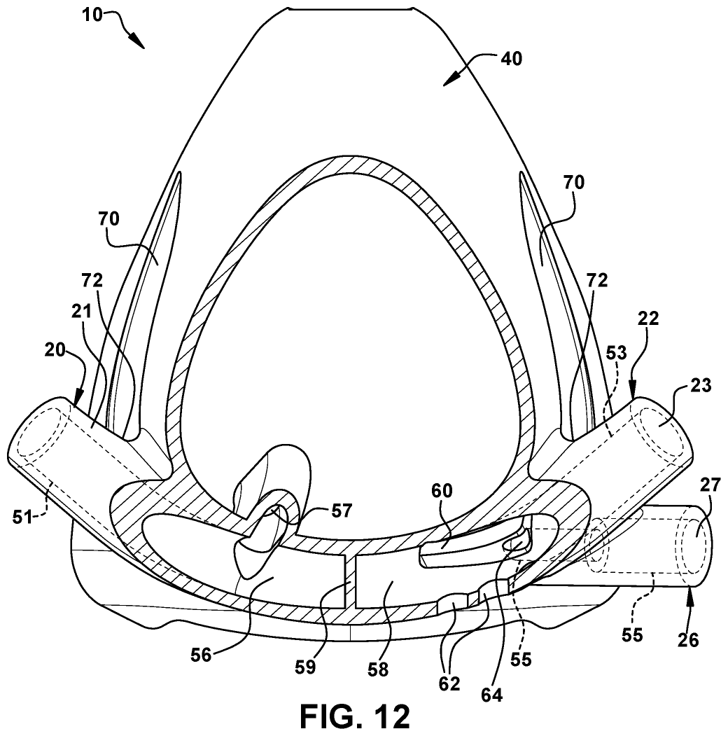

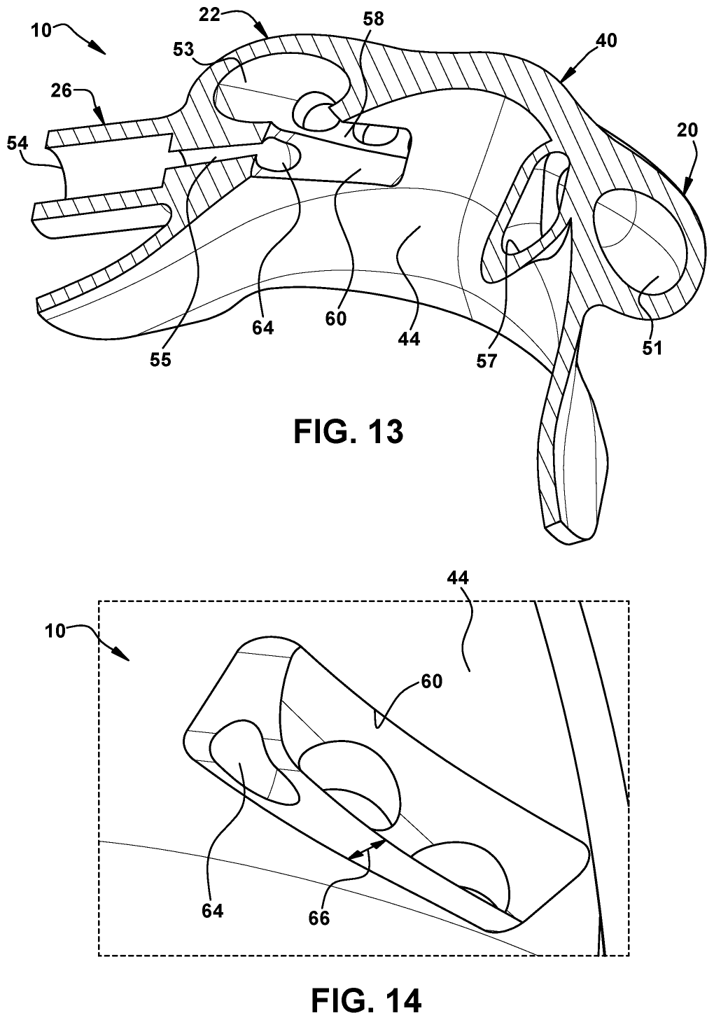

As shown, the first (inflow) tube segment 21 of the inflow port 20 may extend outwardly of the cover portion 42 on a first side of the cover portion 42, such as a right-side of the nose. The inflow tube segment 21 forms an internal inflow fluid passage 51 (FIGS. 12 and 13) that fluidly connects to the external opening 50 of the inflow tube segment 21, which this external opening 50 is an inflow inlet opening (also referred to with 50) configured to receive gas from the inflow (supply) tubing 18 connected thereto (FIG. 1). The inflow tube segment 21 of the inflow port 20 may have any suitable shape and size, which in the illustrated embodiment is a cylindrical tube segment with the internal inflow fluid passage 51 centrally located along the axis of the inflow tube segment 21 and having the inflow inlet opening 50 at an end of the inflow tube segment 21.

The internal inflow fluid passage 51 of the inflow port 20 may fluidly connect to a downstream enlarged enclosed volume that forms an inflow fluid chamber 56 (as best shown in FIG. 12). The inflow fluid chamber 56 is arranged beneath the patient's nose (e.g., at a lower portion of the mask body 40, below the protruding part of the cover portion 42), and includes an inflow outlet 57 for delivering the supply gas to the breathing chamber. As shown in the illustrated embodiment (e.g., FIG. 12), the inflow outlet 57 may be formed as a duct or prong that is configured to direct the inflow gas toward the patient's airway openings in the nostrils. This supply gas can then be breathed in from the breathing cavity 44 by the patient for delivery of the supply gas, such as the anesthesia gas (e.g., N₂O/O₂).

The second (outflow) tube segment 23 of the outflow port 22 may extend outwardly of the cover portion 42 on a second side of the cover portion 42 that is opposite the inflow tube segment 21, such as a left-side of the nose. The outflow tube segment 23 forms an internal outflow fluid passage 53 (FIGS. 12 and 13) that fluidly connects to the external opening 52 of the outflow tube segment 23, which this external opening 52 is an outflow outlet opening (also referred to with 52) configured to deliver expired gas to the outflow (exhalation) tubing 24 connected thereto (FIG. 1). The outflow tube segment 23 of the outflow port 22 may have any suitable shape and size, which in the illustrated embodiment is a cylindrical tube segment with the internal outflow fluid passage 53 centrally located along the axis of the outlet tube segment 23 and having the outlet opening 52 at an end of the outflow tube segment 23.

The internal outflow fluid passage 53 may fluidly connect to an upstream enlarged enclosed volume that forms an outflow fluid chamber 58 (as best shown in FIG. 12). The outflow fluid chamber 58 is arranged beneath the patient's nose adjacent to the inflow fluid chamber 56, which is separated by a divider wall or partition 59. The partition 59 maintains separation between the supply gas system and the output vacuum. The outflow fluid chamber 58 includes an outflow inlet 60 for delivering the expired gas of the patient from the breathing chamber 44 to the outflow fluid chamber. As shown in the illustrated embodiment (e.g., FIGS. 12-14), the outflow inlet 60 may be formed as a window that is arranged below the nose and is suitably sized to collect the expired gas, such as with the aid of vacuum pressure. The outflow fluid chamber 58 also may include openings 62, such as suitably sized apertures, which are configured to scavenge any supply gas (e.g., N₂O/O₂) that may have escaped from within the mask 10 to the work area and/or from exhalant from the patient's mouth.

The third (capnography) tube segment 27 of the capnography port 26 may extend outwardly of the cover portion 42 on the same side as the outflow port 22, such as a left-side of the nose. The capnography tube segment 27 forms an internal capnography fluid passage 55 (FIGS. 12 and 13) that fluidly connects to the external opening 54 of the capnography tube segment 27, which this external opening 27 is a capnography outlet opening (also referred to with 54) configured to deliver a sample of the expired gas to the sampling (capnography) tubing 28 connected thereto (FIG. 1). The capnography tube segment 27 of the capnography port 26 may have any suitable shape and size, which in the illustrated embodiment is a cylindrical tube segment, similarly to the inflow and outflow tube segments 21, 23, with the internal capnography fluid passage 53 centrally located along the axis of the capnography tube segment 23 and having the capnography outlet opening 54 at an end of the capnography tube segment 23.

At least one unique advantage of unitarily forming the capnography port 26 with a portion of the mask body 40 is that the capnography port 26 is closer to the exhalation source of the patient. This provides a means of collecting exhalation gas samples essentially directly from the patient's expired gases, which improves capnography sensing and monitoring. In other words, this unique arrangement of the capnography port 26 provides a non-diluted sensing of the expired gas, which improves monitoring fluctuations and reading by a medical care professional. The integration of the capnography port 26 into the mask body 40 also minimizes the number of components needed to collect the gas samples. This reduces cost and also saves time and the potential for error in assembling the sampling (capnography) tubing to the mask. The fixed location of capnography port 26 integrated into the mask also improves accurate repeatability of using such mask.

To collect the expired gas sample through the capnography port 26 in a direct manner, a capnography inlet opening 64 is arranged proximal the breathing chamber 44 in a region below the patient's nose (e.g., in a lower portion of the mask body 40 that is toward the bottom of the breathing chamber 44 and below the protruding part of the cover portion 42 of the mask body, as shown in FIGS. 12-14, for example). The capnography inlet opening 64 fluidly connects to the internal capnography fluid passage 55 to collect and deliver the sample of expired air/gas through the internal capnography passage 55 and through the capnography outlet opening 52 to the capnography tubing 28 for analysis by the capnography machine 14 (FIG. 1). The capnography inlet opening 64 may have any suitable shape or size to collect the sample of expired gas, such as with the aid of vacuum suction from the capnography machine 14.

In exemplary embodiments, the capnography inlet opening 64 is located downstream of the breathing cavity 44 formed by the internal surface of the cover portion 42. Such a location of the capnography inlet opening 64 downstream of the breathing cavity 44 is advantageous in that it does not disrupt the natural flow of gas through the breathing cavity. In addition, the capnography inlet opening 64 may be located upstream of the outflow inlet opening 60 of the outflow port 22. Such a location of the capnography inlet opening 64 upstream of the edge that defines the outflow inlet opening 60 of the outflow port 22 is advantageous in that any vacuum suction through the capnography port 26 (from the capnography machine 14) should not be adverse to any vacuum suction through the outflow port 22 (from the gas supply machine 16, or other external vacuum, for example).

As best shown in FIGS. 12-14, the capnography inlet opening 64 may be integrally formed in a wall 66 of the mask body 40 that forms the breathing cavity 44. By integrating the capnography inlet opening 64 into the mask body 40, the collection point of the expired gas sample will be in a repeatable and accurate location for each mask use. As shown in the illustrated embodiment, the capnography inlet opening 64 may be formed in the wall 66 of the mask body that also forms the outflow inlet 60 (e.g., window), at a location between the edge of the breathing cavity 44 and the edge of the outflow chamber 58. The thickness of the wall 66 may be in a range from 1 mm to 10 mm (as shown by the double arrow), for example, and thus placement of the capnography inlet opening 64 within the wall 66 and inside of the outflow inlet 60 (window) places the capnography inlet opening 64 as close as practical to the breathing chamber 44 for direct sampling, but without interrupting gas flow in the breathing chamber 44 and/or gas flow to the outflow port 22. For example, a distance from the capnography inlet opening 64 to the edge of breathing cavity 44 may be in a range from 0.25-mm to 5-mm, and the distance from the capnography inlet opening 64 to the edge of the outflow fluid chamber 58 may be the same.

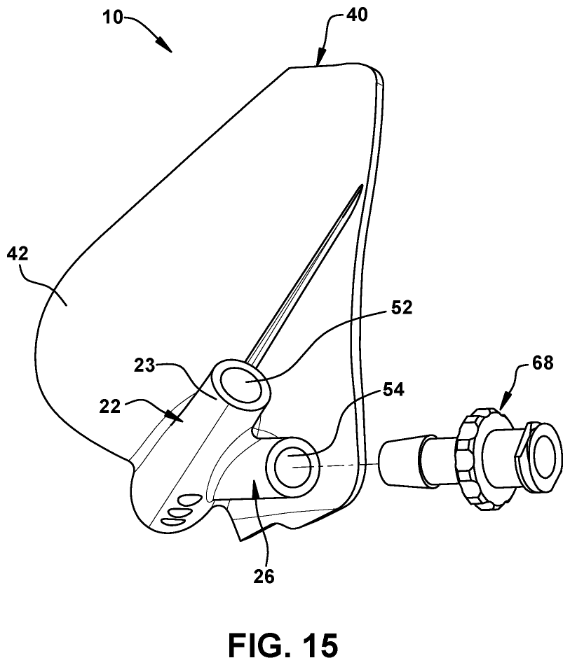

Turning to FIG. 15, the capnography mask 10 may be used with a capnography connector 68 that can be inserted directly into the external (capnography) outlet opening 54 of the capnography port 26. The capnography connector 68 provides a low-profile design to be utilized with the mask 10. As shown, the capnography connector 68 may have a tapered or barb style connection that aids in the simplicity and speed of modularity of the mask, whereby the sampling (capnography) tubing 28 is connected to the inlet of the capnography connector 68. The capnography connector 68 can be connected after the patient is already using the mask 10 with gas flowing through the breathing circuit, as the location of the capnography inlet opening 64 permits an uninterrupted flow of gas to and from the breathing cavity 44. This allows the patient to remain in a state of comfort during the procedure without interruption. It is understood that the capnography connector 68 need not be used, and the unique arrangement of the integrated capnography port 26 also permits direct connection with tubing 28 being inserted into the external capnography outlet opening 54.

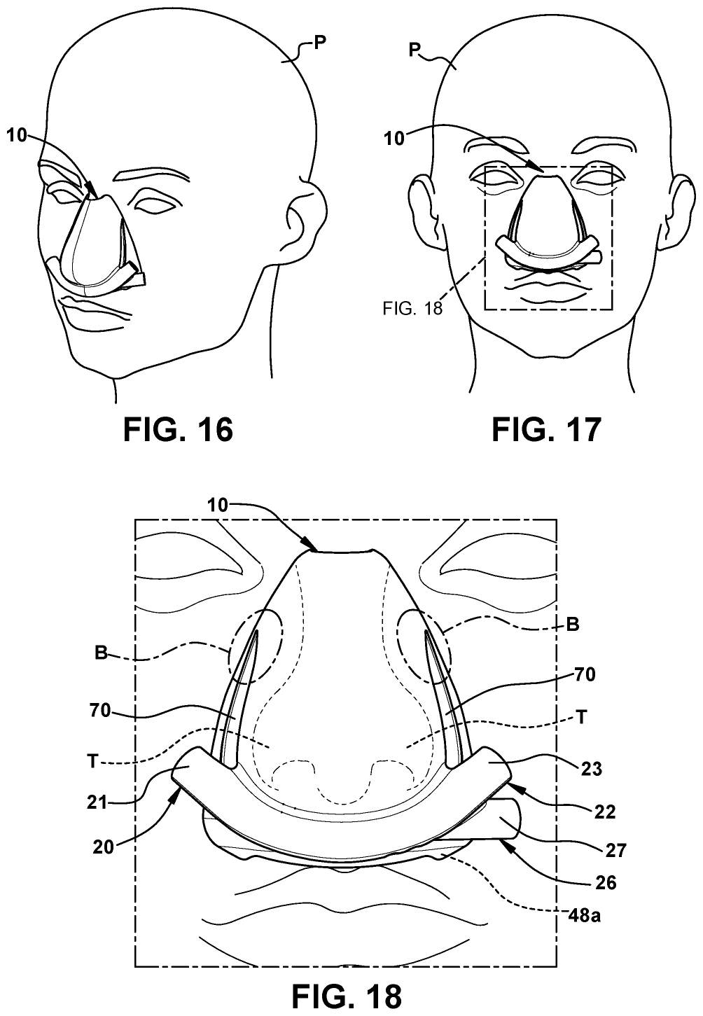

Turning to FIGS. 16-19, the exemplary capnography mask 10 is shown arranged on the patient's face and substantially covering the patient's nose. As noted above, the unique capnography mask 10 may have a structural arrangement that distributes load in way that improves comfort to the patient P and/or improves sealing against the patient's face.

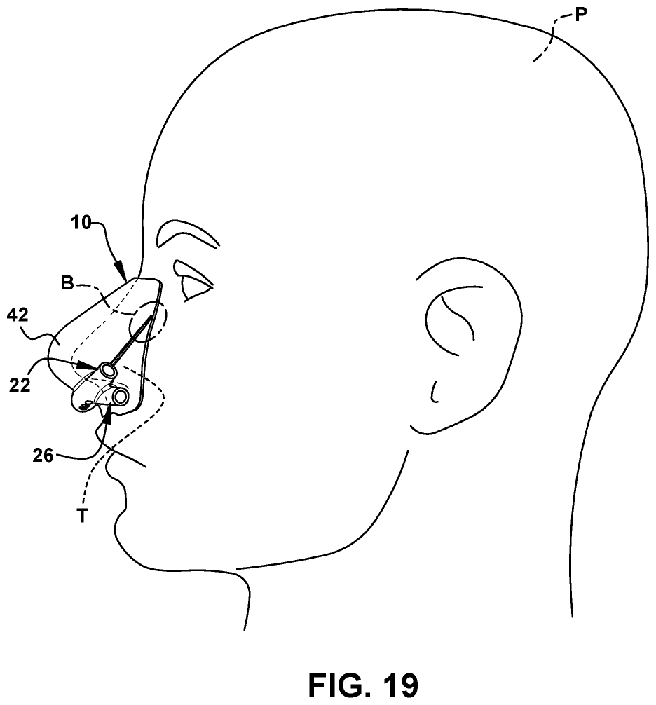

For example, referring particularly to FIGS. 18 and 19, with reference still to FIGS. 2-15, the exemplary capnography mask 10 may include one or more external ribs 70 that are oriented in such a way that load from the mask 10 is distributed away from the alar fibrofatty tissue T of the nose to regions B of the patient's face that are above the alar fibrofatty tissue T. The exemplary load regions B above the alar fibrofatty tissue T may include the upper boney regions of the patient's face, including, for example, the nasal bone, nasal cartilage, and/or the maxilla of the face. Distributing more load to these upper boney regions B, and not on the alar fibrofatty tissue T, improves comfort to the patient P by not pinching the nostrils toward closed.

Alternatively or additionally to the ribs 70, the orientation of the inflow tube segment 21 of the inflow port 20 and the orientation of the outflow tube segment 23 of the outflow port 22 also may be arranged in such a way that load from the mask 10 is distributed away from the alar fibrofatty tissue T to the regions B of the patient's face that are above the alar fibrofatty tissue T. In exemplary embodiments, the arrangement of the ribs 70 and the tube segments 21, 23 may cooperate with each other to provide such an improved distribution of load. The load at the regions B above the alar fibrofatty tissue T may constitute a majority of the overall load from the mask. The improved distribution of load also may improve sealability around the peripheral sealing edge 48 of the mask against the patient's face.

Turning to FIGS. 20A-20D, various exemplary sizes of masks are shown including adult large (FIG. 20A), adult medium (FIG. 20B), adult small (FIG. 20C), and pediatric/child (FIG. 20D). As shown in these illustrations, the outflow tube segment 23 of the outflow port 22 extends at an upward angle (α) to distribute more load to the regions B above the alar fibrofatty tissue T (e.g., the upper boney region including the nasal bone, nasal cartilage, and/or maxilla of the face), as compared with load from the mask applied to the alar fibrofatty tissue T, if any such alar fibrofatty tissue load exists at all.

As shown, the outflow tube segment 23 may extend from below the protruding part of the cover portion 42 of the mask 10 at an angle (α) that is upward and rearward. The desired angle (α) may be determined experimentally, such as via finite element analysis (FEA) or by trial-and-error. In exemplary embodiments, it is found that an angle (α) in a range from about 15-degrees to about 55-degrees, more particularly about 25-degrees to about 45-degrees, more particularly from about 30-degrees to about 40-degrees (including all ranges and subranges between the stated values), is sufficient to distribute load away from the alar fibrofatty tissue T to the regions B of the face and/or nose above the alar fibrofatty tissue T. In the illustrated embodiment, the angle (α) is about 33-degrees. As shown, the relatively steep angle α is such that the outflow tube segment 23 is oriented above the capnography tube segment 27 of the capnography port 26 on the same side of the nose. Although not shown in FIGS. 20A-20D, it is understood that the inflow tube segment 21 of the inflow port 20 is a mirror image of the outflow tube segment 23 of the outflow port 22, and therefore the inflow tube segment 21 extends at the same angle (α) on the opposite side of the nose.

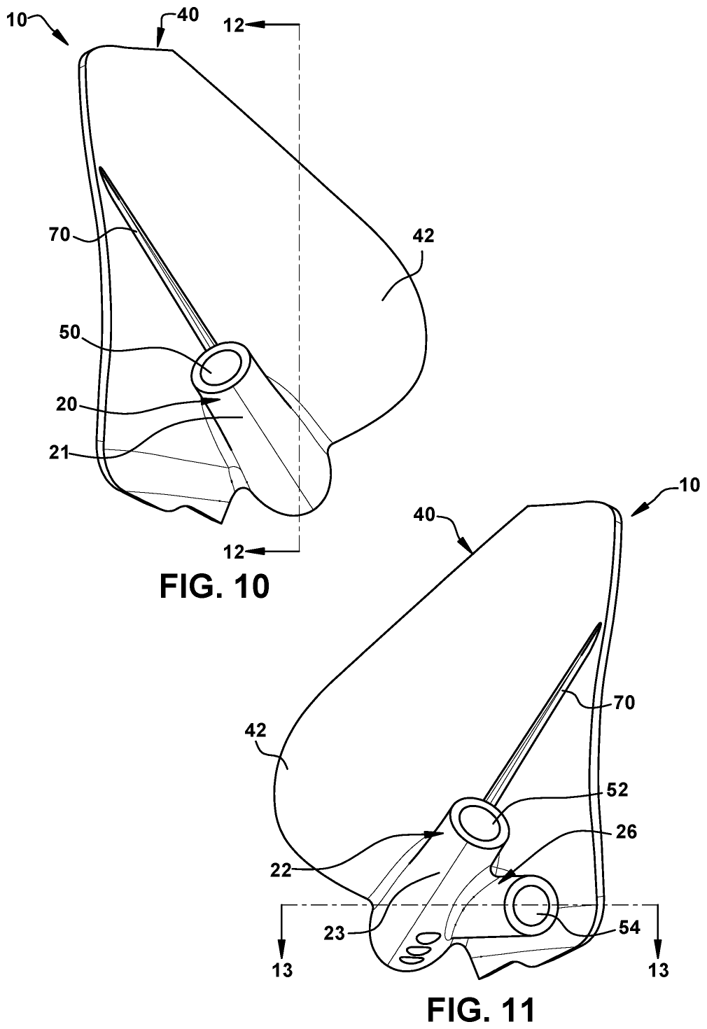

Also with reference to FIGS. 20A-20D, and also to FIGS. 10 and 11, the ribs 70 on each side of the mask 10 may extend at about the same angle (α) as the respective outflow and inflow tube segments 21, 23 to distribute more load to the regions B above the alar fibrofatty tissue T, as compared with load from the mask applied to the alar fibrofatty tissue T, if any. In the illustrated embodiment, for example, the respective ribs 70 are at about the same angle as the tube segments 21, 23 by being only slightly offset by about 2-degrees from the angle of the tube segments 21, 23. As such, it is found that an angle (α) in a range from about 10-degrees to about 65-degrees, more particularly from about 20-degrees to about 55-degrees, more particularly from about 27-degrees to about 42-degrees (including all ranges and subranges between the stated values), is sufficient to distribute load away from the alar fibrofatty tissue T to the regions B above the alar fibrofatty tissue T. In the illustrated embodiment, for example, the ribs 70 each extend at an inclined angle of about 34-degrees. It is understood that although the ribs 70 are at about the same angle (α) as the tube segments 21, 23 (such as the ribs 70 being offset in a range from 0-degrees to 10-degrees from the angle of the tube segments 21,23), the ribs 70 could be at a different angle depending on the angle of the tube segments 21, 23 to distribute of the load away from the alar fibrofatty tissue T.

Referring back particularly to the enlarged view of FIG. 18, the ribs 70 on each side of the mask 10 may be arranged to cooperate with the respective inflow and outflow tube segments 21, 23 to provide the desired distribution of load to the regions B above the alar fibrofatty tissue T. In the illustrated embodiment, for example, each rib 70 starts at the intersection of the respective tube segment 21, 23 with the cover portion 42 of the mask as designated at 72. The ribs 70 extend upwardly and toward the face at the angle (α), but instead of diverging laterally like the tube segments 21, 23, the ribs 70 angle around the alar fibrofatty tissue T, therefore distributing the load away from the alar fibrofatty tissue T. The ribs 70 terminate at the upper portion of the mask 10, near the periphery as designated at 74, which is at or near the region B where the load is distributed to the patient's face and/or nose. In this manner, the ribs 70 essentially provide bridges that span around the alar fibrofatty tissue T to distribute load from the tube segments 21, 23 to the upper boney regions B of the patient's face.

The structural support from the ribs 70 cooperating with the tube segments 21, 23 of the inflow and outflow ports 20, 22 also may improve the seal at the sealing edge 48 around the patient's nose to prevent gas leaking into the external area around the mask. This occurs in the illustrated embodiment by the ribs 70 pulling the mask 10 upwardly such that the lower sealing edge 48a seals against the upper lip region (philtrum) and distributes load around the alar fibrofatty tissue T and inwardly to the upper boney region B to provide a good seal. Such an improved seal also may reduce or eliminate the need for adhesives, thereby providing a more consistent user experience that is not affected by repositioning the mask 10.

Turning now to FIG. 21, another exemplary embodiment of a capnography mask 110 is shown. The capnography mask 110 is substantially the same as the above-referenced capnography mask 10, except that the capnography mask 110 includes a mouth covering portion 175 below the nose covering portion 142. Consequently, the same reference numerals but indexed by 100 are used to denote structures corresponding to similar structures in the capnography masks 10, 110, including the mask body 140, cover portion 142, ribs 170, inflow port 120, outflow port 122, and capnography port 146 that are all integral and unitary with the mask body 140. The mouth covering portion 175 also may be integral and unitary with the mask body 140. As such, the foregoing description of the capnography mask 10 is equally applicable to the capnography mask 110, and it is understood that aspects of the capnography masks 10, 110 may be substituted for one another or used in conjunction with one another where applicable.

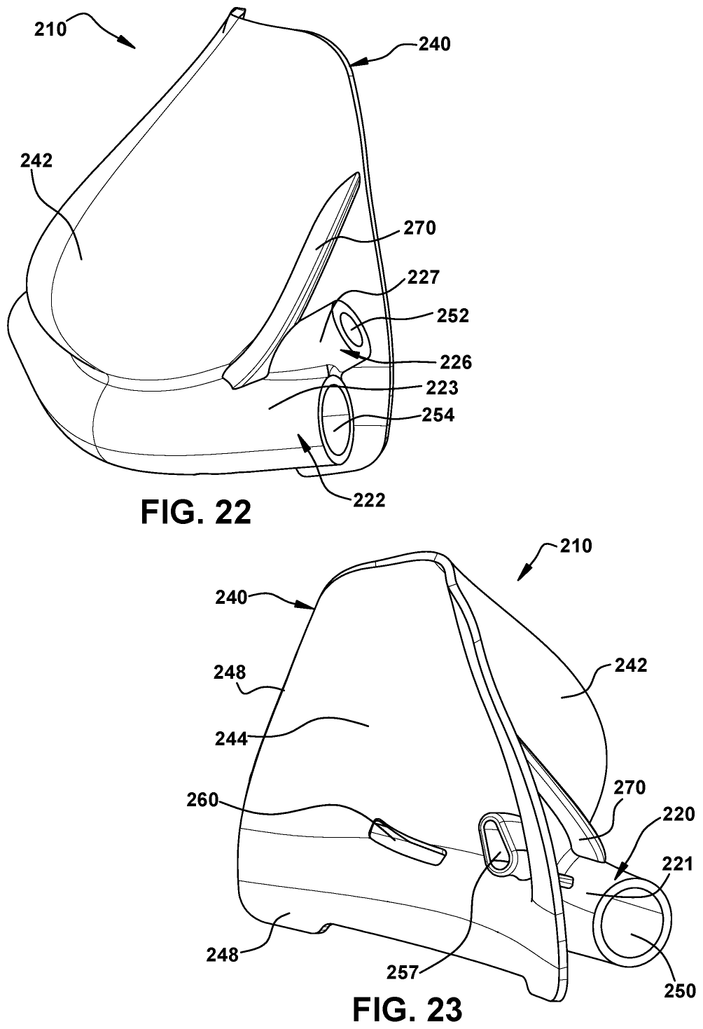

Turning to FIGS. 22-26, another exemplary embodiment of a capnography mask 210 is shown. The capnography mask 210 is substantially similar to the above-referenced capnography mask 10, and consequently the same reference numerals but in the 200-series are used to denote structures corresponding to similar structures in the capnography masks. Accordingly, the foregoing description of the capnography mask 10 is equally applicable to the capnography mask 210, except as noted below. It is also understood that aspects of the capnography masks 10, 210 may be substituted for one another or used in conjunction with one another where applicable.

As shown, similarly to the mask 10, the capnography mask 210 includes a mask body 240, a cover portion 242 forming a breathing cavity 244, an inflow port 220, outflow port 222, and capnography port 226 that are all integral and unitary with the mask body 240. The ports 220, 222, 226 are formed as respective tube segments 221, 223, 227 that extend outwardly of the cover portion 242 of the mask body 240, and one or more ribs 270 may be provided to distribute load from the mask 210 toward a region of the patient's face that is above the alar fibrofatty tissue. The mask 210 includes an inflow inlet 250, an internal inflow passage 251, an inflow fluid chamber 256, and an inflow outlet 257. The mask 210 also includes an outflow inlet 260, an outflow fluid chamber 258, an outflow internal fluid passage 253 and an outflow outlet 252. Furthermore, the mask 210 includes a capnography inlet opening 264, a capnography internal fluid passage 255, and a capnography outlet 254.

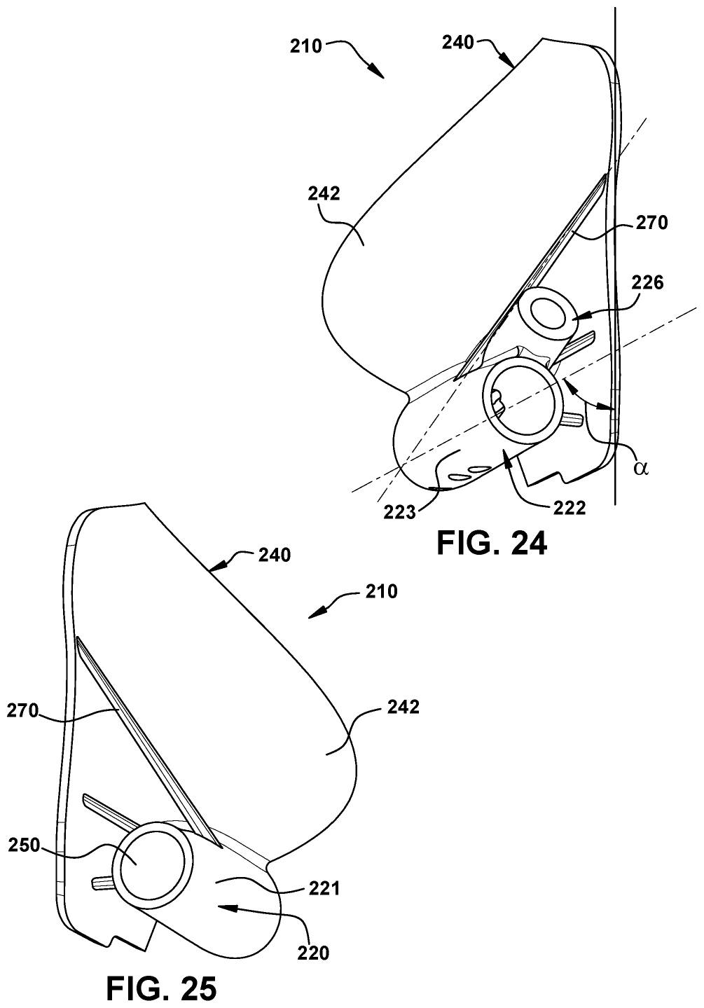

Referring particularly to FIG. 24, one difference with the capnography mask 210 compared to the capnography mask 10 is that the inflow port and outflow port are oriented at a lower angle (α), and the capnography port 226 is located above the outflow port 222. In the illustrated embodiment, for example, this angle (α) is at about 57-degrees. The ribs 270 on the opposite sides of the mask 210 begin at the intersection of the tube segments 221, 223 and end at an upper portion of the mask to distribute load away from the alar fibrofatty tissue, similarly to that of the mask 10 described above. However, it is found that the lower angle (α) orientation of the inflow and outflow tube segments 221, 223 of the mask 210 do not provide as good of a seal against the patient's face as compared to the mask 10 with the higher angle (α) orientation of tube segments 21, 23. As such, the mask 210 may need to use an adhesive at the sealing edge 248 to facilitate sealing performance. On the other hand, it was surprisingly found that when the upward angle (α) of the inflow and outflow tube segments 21, 23 was reduced to less than 57-degrees, more particularly in a range from about 30-degrees to about 40-degrees, as is the case in the exemplary mask 10, this improved load distribution so much that an adhesive was no longer needed to provide an adequate seal against the patient's face.

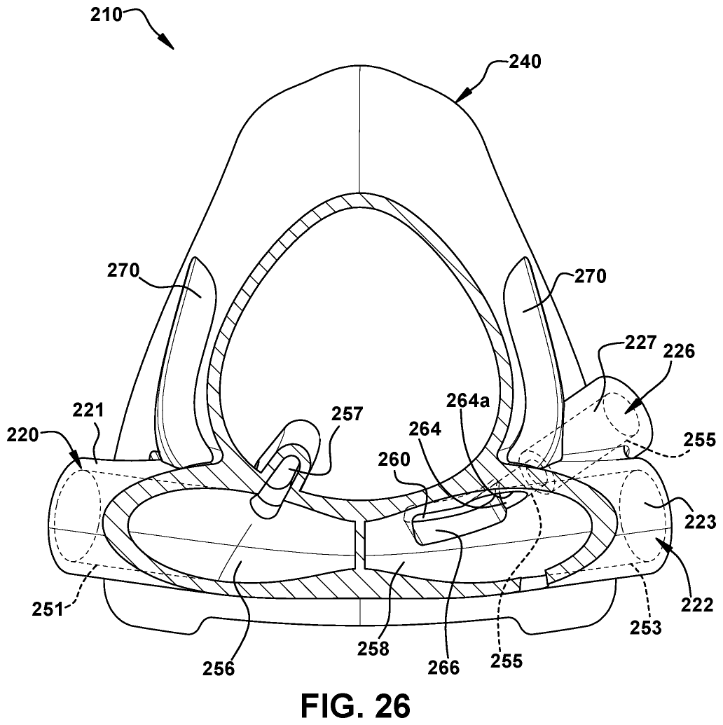

Referring to FIG. 26, another difference with the capnography mask 210 compared to the capnography mask 10 is that the capnography inlet opening 264 is not fully bounded within the wall 266 between the breathing cavity 244 and the outlet fluid chamber 258. Rather, the capnography inlet opening 264 has a notched portion 264a that opens into the outflow fluid chamber 258. Although such a capnography inlet opening 264 is integrated into the mask body 240 and is located proximal to the breathing cavity 244 under the patient's nose to provide the benefits described above, the notched portion 264a opening to the outflow fluid chamber may tend to introduce more interruptions to gas flow as compared to the more fully-bounded capnography inlet opening 64 located between the edges in the wall 66 of the window 60 according to the mask 10 and/or the mask with notched portion 264a may be more difficult to manufacture.

Exemplary capnography mask(s) have been described herein, in which the mask includes a mask body having a cover portion configured to cover at least the nostrils of a patient's nose and forms a breathing cavity for inhalation of inflow gas and exhalation of expired gas. The mask also includes an inflow port fluidly connected to the breathing cavity for delivering the inflow gas to the breathing cavity, an outflow port fluidly connected to the breathing cavity for delivering the expired gas out of the breathing cavity, and a capnography port fluidly connected to the breathing cavity for delivering a sample of the expired gas to a capnography machine. The inflow, outflow and capnography ports may be unitarily formed by respective portions of the mask body to form a unitary mask structure. The ports may include respective tube segments that may cooperate with respective ribs to distribute load away from the alar fibrofatty tissue of the patient's nose, thereby improving comfort.

Exemplary benefits of the unique capnography mask and integral capnography port can be broken down into at least three factors: location, simplicity, and modularity. The mask achieves the goal of cost savings due to simplification and modularity while providing accurate gas measurements of the patients breathing. The location of the capnography port allows direct access to the exhaled gas, particularly CO₂, for better measurements/monitoring of CO₂. Simplicity of the capnography port allows the direct access to the freshly exhaled gas from the nose but maintain the low-profile design of the mask. This design reduces the number of components needed for capnography which then decreases cost and increases manufacturability. The design allows for modularity for the user which would allow the doctor or staff to connect the capnography machine as needed without interrupting the main function of the mask, delivering of fresh gas and vacuuming excess gas

According to an aspect, a capnography mask includes: a mask body having a cover portion that is configured to cover at least the nostrils of a patient's nose and which forms a breathing cavity for inhalation of inflow gas through the patient's nose and exhalation of expired gas through the patient's nose, the mask body further including: an inflow port unitarily formed by a first portion of the mask body, the inflow port being fluidly connected to the breathing cavity for delivering the inflow gas to the breathing cavity; an outflow port unitarily formed by a second portion of the mask body, the outflow port being fluidly connected to the breathing cavity for delivering the expired gas out of the breathing cavity; and a capnography port unitarily formed by a third portion of the mask body, the capnography port being fluidly connected to the breathing cavity for delivering a sample of the expired gas to a capnography machine.

Exemplary embodiments may include one or more of the following additional features, separately or in any combination.

In exemplary embodiments, a capnography inlet opening is unitarily formed by the mask body proximal the breathing chamber in a region below the patient's nose, the capnography inlet opening being fluidly connected to an internal capnography fluid passage of the capnography port, the internal capnography fluid passage being unitarily formed by the mask body.

In exemplary embodiments, the capnography inlet opening is configured to collect the sample of expired gas, and the internal capnography fluid passage being configured to deliver the sample of expired gas through the capnography port for delivery to the capnography machine.

In exemplary embodiments, the capnography inlet opening is located downstream of the breathing cavity.

In exemplary embodiments, the capnography inlet opening is located upstream of the outflow port.

In exemplary embodiments, an outflow opening is formed in a wall of the mask body that forms at least part of the breathing chamber.

In exemplary embodiments, an internal edge of the wall being part of the breathing chamber, and an outer edge of the wall being part of the outflow port.

In exemplary embodiments, the capnography inlet being formed in the wall between the internal edge and the outer edge.

In exemplary embodiments, the inflow port includes an inflow tube segment that extends outwardly of the cover portion of the mask body.

In exemplary embodiments, the outflow port includes an outflow tube segment that extends outwardly of the cover portion of the mask body.

In exemplary embodiments, the capnography port includes a capnography tube segment that extends outwardly of the cover portion of the mask body.

In exemplary embodiments, the inflow tube segment extends outwardly of the cover portion on a first side of the cover portion.

In exemplary embodiments, the outflow tube segment extends outwardly of the cover portion on a second side of the cover portion that is opposite the inflow tube segment.

In exemplary embodiments, the capnography tube segment extends outwardly of the cover portion on the same side of the cover portion as the outflow tube segment.

In exemplary embodiments, the inflow port includes an external inflow inlet opening at an end portion of the inflow tube segment.

In exemplary embodiments, the outflow port includes an external outflow outlet opening at an end portion of the outflow tube segment.

In exemplary embodiments, the capnography port includes an external capnography outlet opening at an end portion of the capnography tube segment.

In exemplary embodiments, an internal inflow fluid passage of the inflow port is unitarily formed by the mask body and is fluidly connected to a downstream inflow fluid chamber that is arranged beneath the patient's nose.

In exemplary embodiments, an inflow outlet is fluidly connected to the inflow fluid chamber and is configured to deliver the inflow gas from the inflow fluid chamber to the breathing chamber.

In exemplary embodiments, an internal outflow fluid passage of the outflow port is unitarily formed by the mask body and is fluidly connected to an upstream outflow fluid chamber that is arranged beneath the patient's nose.

In exemplary embodiments, an outflow inlet is fluidly connected to the outflow fluid chamber and is configured to deliver the expired gas from the breathing chamber to the outflow fluid chamber.

In exemplary embodiments, each of the inflow tube segment, the outflow tube segment, and the capnography tube segment is resiliently flexible.

In exemplary embodiments, the inflow tube segment of the inflow port and the outflow tube segment of the outflow port each extends at an upward angle such that load from the mask is distributed away from the alar fibrofatty tissue of the patient's nose to a region of the patient's face and/or nose that is above the alar fibrofatty tissue when in use.

In exemplary embodiments, the mask body includes a first rib that intersects with the inflow tube segment and extends upwardly and rearwardly around the alar fibrofatty tissue on a first side of the nose and terminates at a first upper region on the first side of the mask to cooperate with the inflow tube segment thereby distributing load away from the alar fibrofatty tissue on the first side to a first region of the patient's face and/or nose above the alar fibrofatty tissue on the first side.

In exemplary embodiments, the mask body includes a second rib that intersects with the outflow tube segment and extends upwardly and rearwardly around the alar fibrofatty tissue on a second side of the nose and terminates at a second upper region on the second side of the mask to cooperate with the outflow tube segment thereby distributing load away from the alar fibrofatty tissue on the second side to a second region of the patient's face and/or nose above the alar fibrofatty tissue on the second side.

In exemplary embodiments, the mask body includes a rib arrangement that distributes load from the mask away from the alar fibrofatty tissue of the patient's nose to a region of the patient's face and/or nose that is above the alar fibrofatty tissue when in use.

In exemplary embodiments, the mask body is made with a flexible, resilient material in which the cover portion is molded into a nose-conforming shell that substantially covers the patient's nose with a peripheral sealing edge that seals against the patient's face.

In exemplary embodiments, the mask body is formed as a single unitary structure formed by injection molding or additive manufacturing.

In exemplary embodiments, the mask body further includes a portion that covers the patient's mouth.

According to another aspect, a capnography mask includes: a mask body having a cover portion that is configured to cover at least the nostrils of a patient's nose and which forms a breathing cavity for inhalation of inflow gas through the patient's nose and exhalation of expired gas through the patient's nose; an inflow port formed by a first tube segment that extends outwardly of the cover portion on a first side of the cover portion, the inflow port being fluidly connected to the breathing cavity for delivering the inflow gas to the breathing cavity; and an outflow port formed by a second tube segment that extends outwardly of the cover portion on a second side of the cover portion that is opposite the first side, the outflow port being fluidly connected to the breathing cavity for delivering the expired gas out of the breathing cavity; wherein the first tube segment of the inflow port and the second tube segment of the outflow port each extends at an upward angle such that load from the mask is distributed away from the alar fibrofatty tissue of the patient's nose to a region of the patient's face and/or nose that is above the alar fibrofatty tissue when in use.

Exemplary embodiments may include one or more of the following additional features, separately or in any combination.

In exemplary embodiments, the mask further comprising: a capnography port formed by a third tube segment that extends outwardly of the cover portion on the second side of the cover portion, the capnography port being fluidly connected to the breathing cavity for delivering a sample of the expired gas to a capnography machine.

In exemplary embodiments, the first, second and third tube segments are each unitarily formed by respective portions of the mask body to form a resiliently flexible and unitary mask structure.

In exemplary embodiments, the first and second tube segments each start below the patient's nose and extend upwardly and rearwardly in which the upward angle is relative to a vertical plane of the mask body and is in a range from 15-degrees to 55-degrees.

In exemplary embodiments, the mask body includes a first rib that intersects with the first tube segment and extends upwardly and rearwardly around the alar fibrofatty tissue on a first side of the nose and terminates at a first upper region on the first side of the mask to cooperate with the first tube segment thereby distributing load away from the alar fibrofatty tissue on the first side to a first region of the patient's face and/or nose above the alar fibrofatty tissue on the first side.

In exemplary embodiments, the mask body includes a second rib that intersects with the second tube segment and extends upwardly and rearwardly around the alar fibrofatty tissue on a second side of the nose and terminates at a second upper region on the second side of the mask to cooperate with the second tube segment thereby distributing load away from the alar fibrofatty tissue on the second side to a second region of the patient's face and/or nose above the alar fibrofatty tissue on the second side.

According to another aspect, a capnography mask includes: a mask body having a cover portion that is configured to cover at least the nostrils of a patient's nose and which forms a breathing cavity for inhalation of inflow gas through the patient's nose and exhalation of expired gas through the patient's nose; an inflow port fluidly connected to the breathing cavity for delivering the inflow gas to the breathing cavity; an outflow port fluidly connected to the breathing cavity for delivering the expired gas out of the breathing cavity; and a rib arrangement configured to distribute load away from the alar fibrofatty tissue of the patient's nose to a region of the patient's face and/or nose that is above the alar fibrofatty tissue when in use.

Exemplary embodiments may include one or more of the following additional features, separately or in any combination.

In exemplary embodiments, the rib arrangement includes a first rib on a first side of the mask and a second rib on a second side of the mask opposite the first side, the first and second ribs each extending at an upward angle such that load from the mask is distributed away from the alar fibrofatty tissue of the patient's nose to a region of the patient's face and/or nose that is above the alar fibrofatty tissue when in use.

In exemplary embodiments, the respective upward angles of the first and second ribs are each in a range from 10-degrees to 65-degrees.

In exemplary embodiments, the inflow port includes an inflow tube segment that extends outwardly of the cover portion of the mask body.

In exemplary embodiments, the outflow port includes an outflow tube segment that extends outwardly of the cover portion of the mask body.

In exemplary embodiments, the first rib intersects with the inflow tube segment and extends upwardly and rearwardly around the alar fibrofatty tissue on a first side of the nose and terminates at a first upper region on the first side of the mask to cooperate with the inflow tube segment thereby distributing load away from the alar fibrofatty tissue on the first side to a first region of the patient's face and/or nose above the alar fibrofatty tissue on the first side.

In exemplary embodiments, the second rib that intersects with the outflow tube segment and extends upwardly and rearwardly around the alar fibrofatty tissue on a second side of the nose and terminates at a second upper region on the second side of the mask to cooperate with the outflow tube segment thereby distributing load away from the alar fibrofatty tissue on the second side to a second region of the patient's face and/or nose above the alar fibrofatty tissue on the second side.

In exemplary embodiments, the mask further comprising: a capnography port formed by a third tube segment that extends outwardly of the cover portion on the second side of the cover portion, the capnography port being fluidly connected to the breathing cavity for delivering a sample of the expired gas to a capnography machine.

According to another aspect, a capnography system includes: the capnography mask according to any of the foregoing or following, in which the inflow port and the outflow port are connected to a breathing circuit with tubing, and the capnography port is connected to a capnography machine with tubing.

In exemplary embodiments, the breathing circuit includes a gas supply regulator that supplies the inflow gas to the inflow port via inflow tubing, and a vacuum source pulls the expired gas through the outflow port, and the capnography machine includes a separate vacuum source that pulls the sample of the expired gas through the capnography port.

According to another aspect, a mask includes: an injection molded mask body adapted to substantially cover a patient's nose and mouth and having a peripheral edge adapted to substantially seal against the patient's face and forming a compartment; an inflow port defined in the mask body; an outflow port defined in the mask body; and a capnography port defined in the mask body.

Exemplary embodiments may include one or more of the following additional features, separately or in any combination.

In exemplary embodiments, the mask further includes a capnography connector removably positionable in the capnography port of the mask body.

In exemplary embodiments, the mask body comprises at least one rib protruding from an exterior side of the mask body.

In exemplary embodiments, the rib is formed starting at an intersection of a tube defining the inflow port and outflow port and a nose cover portion of the mask and proceeding at an angle away from the alar fibrofatty tissue of a user.

Other exemplary embodiments may include any of the foregoing aspects or embodiments in combination with any aspect or embodiment.

It is to be understood that terms such as “top,” “bottom,” “upper,” “lower,” “left,” “right,” “front,” “rear,” “forward,” “rearward,” and the like as used herein may refer to an arbitrary frame of reference, rather than to the ordinary gravitational frame of reference.

It is to be understood that all ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The term “about” as used herein refers to any value which lies within the range defined by a variation of up to ±10% of the stated value, for example, +10%, ±9%, ±8%, ±7%, ±6%, ±5%, ±4%, ±3%, ±2%, ±1%, ±0.01%, or ±0.0% of the stated value, as well as values intervening such stated values.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The transitional words or phrases, such as “comprising,” “including,” “carrying,” “having,” “containing,” “involving,” “holding,” and the like, are to be understood to be open-ended, i.e., to mean including but not limited to.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A capnography mask comprising:
a mask body having a cover portion that is configured to cover at least the nostrils of a patient's nose and which forms a breathing cavity for inhalation of inflow gas through the patient's nose and exhalation of expired gas through the patient's nose, the mask body further comprising:
an inflow port unitarily formed by a first portion of the mask body, the inflow port being fluidly connected to the breathing cavity for delivering the inflow gas to the breathing cavity;
an outflow port unitarily formed by a second portion of the mask body, the outflow port being fluidly connected to the breathing cavity for delivering the expired gas out of the breathing cavity; and
a capnography port unitarily formed by a third portion of the mask body, the capnography port being fluidly connected to the breathing cavity for delivering a sample of the expired gas to a capnography machine.

2. The capnography mask according to claim 1, wherein:
a capnography inlet opening is unitarily formed by the mask body proximal the breathing chamber in a region below the patient's nose, the capnography inlet opening being fluidly connected to an internal capnography fluid passage of the capnography port, the internal capnography fluid passage being unitarily formed by the mask body,
the capnography inlet opening being configured to collect the sample of expired gas, and the internal capnography fluid passage being configured to deliver the sample of expired gas through the capnography port for delivery to the capnography machine.

3-4. (canceled)

5. The capnography mask according to claim 2, wherein:
an outflow opening is formed in a wall of the mask body that forms at least part of the breathing chamber,
an internal edge of the wall being part of the breathing chamber, and an outer edge of the wall being part of the outflow port, and
the capnography inlet being formed in the wall between the internal edge and the outer edge.

6. The capnography mask according to claim 1, wherein:
the inflow port includes an inflow tube segment that extends outwardly of the cover portion of the mask body;
the outflow port includes an outflow tube segment that extends outwardly of the cover portion of the mask body; and
the capnography port includes a capnography tube segment that extends outwardly of the cover portion of the mask body.

7. The capnography mask according to claim 6, wherein:
the inflow tube segment extends outwardly of the cover portion on a first side of the cover portion,
the outflow tube segment extends outwardly of the cover portion on a second side of the cover portion that is opposite the inflow tube segment, and
the capnography tube segment extends outwardly of the cover portion on the same side of the cover portion as the outflow tube segment.

8. The capnography mask according to claim 6, wherein:
the inflow port includes an external inflow inlet opening at an end portion of the inflow tube segment;
the outflow port includes an external outflow outlet opening at an end portion of the outflow tube segment; and
the capnography port includes an external capnography outlet opening at an end portion of the capnography tube segment.

9. The capnography mask according to claim 1, wherein:
an internal inflow fluid passage of the inflow port is unitarily formed by the mask body and is fluidly connected to a downstream inflow fluid chamber that is arranged beneath the patient's nose, and
an inflow outlet is fluidly connected to the inflow fluid chamber and is configured to deliver the inflow gas from the inflow fluid chamber to the breathing chamber.

10. The capnography mask according to claim 1, wherein:
an internal outflow fluid passage of the outflow port is unitarily formed by the mask body and is fluidly connected to an upstream outflow fluid chamber that is arranged beneath the patient's nose, and
an outflow inlet is fluidly connected to the outflow fluid chamber and is configured to deliver the expired gas from the breathing chamber to the outflow fluid chamber.

11-12. (canceled)

13. The capnography mask according to claim 6, wherein:
the mask body includes a first rib that intersects with the inflow tube segment and extends upwardly and rearwardly around the alar fibrofatty tissue on a first side of the nose and terminates at a first upper region on the first side of the mask to cooperate with the inflow tube segment thereby distributing load away from the alar fibrofatty tissue on the first side to a first region of the patient's face and/or nose above the alar fibrofatty tissue on the first side; and
the mask body includes a second rib that intersects with the outflow tube segment and extends upwardly and rearwardly around the alar fibrofatty tissue on a second side of the nose and terminates at a second upper region on the second side of the mask to cooperate with the outflow tube segment thereby distributing load away from the alar fibrofatty tissue on the second side to a second region of the patient's face and/or nose above the alar fibrofatty tissue on the second side.

14-17. (canceled)

18. A capnography mask comprising:
a mask body having a cover portion that is configured to cover at least the nostrils of a patient's nose and which forms a breathing cavity for inhalation of inflow gas through the patient's nose and exhalation of expired gas through the patient's nose;
an inflow port formed by a first tube segment that extends outwardly of the cover portion on a first side of the cover portion, the inflow port being fluidly connected to the breathing cavity for delivering the inflow gas to the breathing cavity; and
an outflow port formed by a second tube segment that extends outwardly of the cover portion on a second side of the cover portion that is opposite the first side, the outflow port being fluidly connected to the breathing cavity for delivering the expired gas out of the breathing cavity;
wherein the first tube segment of the inflow port and the second tube segment of the outflow port each extends at an upward angle such that load from the mask is distributed away from the alar fibrofatty tissue of the patient's nose to a region of the patient's face and/or nose that is above the alar fibrofatty tissue when in use.

19. The capnography mask according to claim 18, further comprising:
a capnography port formed by a third tube segment that extends outwardly of the cover portion on the second side of the cover portion, the capnography port being fluidly connected to the breathing cavity for delivering a sample of the expired gas to a capnography machine.

20. The capnography mask according to claim 19, wherein:
the first, second and third tube segments are each unitarily formed by respective portions of the mask body to form a resiliently flexible and unitary mask structure.

21. The capnography mask according to claim 18, wherein:
the first and second tube segments each start below the patient's nose and extend upwardly and rearwardly in which the upward angle is relative to a vertical plane of the mask body and is in a range from 15-degrees to 55-degrees.

22. The capnography mask according to claim 18, wherein:
the mask body includes a first rib that intersects with the first tube segment and extends upwardly and rearwardly around the alar fibrofatty tissue on a first side of the nose and terminates at a first upper region on the first side of the mask to cooperate with the first tube segment thereby distributing load away from the alar fibrofatty tissue on the first side to a first region of the patient's face and/or nose above the alar fibrofatty tissue on the first side; and
the mask body includes a second rib that intersects with the second tube segment and extends upwardly and rearwardly around the alar fibrofatty tissue on a second side of the nose and terminates at a second upper region on the second side of the mask to cooperate with the second tube segment thereby distributing load away from the alar fibrofatty tissue on the second side to a second region of the patient's face and/or nose above the alar fibrofatty tissue on the second side.

23. A capnography mask comprising:
a mask body having a cover portion that is configured to cover at least the nostrils of a patient's nose and which forms a breathing cavity for inhalation of inflow gas through the patient's nose and exhalation of expired gas through the patient's nose;
an inflow port fluidly connected to the breathing cavity for delivering the inflow gas to the breathing cavity;
an outflow port fluidly connected to the breathing cavity for delivering the expired gas out of the breathing cavity; and
a rib arrangement configured to distribute load away from the alar fibrofatty tissue of the patient's nose to a region of the patient's face and/or nose that is above the alar fibrofatty tissue when in use.

24. The capnography mask according to claim 23, wherein:
the rib arrangement includes a first rib on a first side of the mask and a second rib on a second side of the mask opposite the first side, the first and second ribs each extending at an upward angle such that load from the mask is distributed away from the alar fibrofatty tissue of the patient's nose to a region of the patient's face and/or nose that is above the alar fibrofatty tissue when in use.

25. (canceled)

26. The capnography mask according to claim 23, wherein:
the inflow port includes an inflow tube segment that extends outwardly of the cover portion of the mask body;
the outflow port includes an outflow tube segment that extends outwardly of the cover portion of the mask body;
the first rib intersects with the inflow tube segment and extends upwardly and rearwardly around the alar fibrofatty tissue on a first side of the nose and terminates at a first upper region on the first side of the mask to cooperate with the inflow tube segment thereby distributing load away from the alar fibrofatty tissue on the first side to a first region of the patient's face and/or nose above the alar fibrofatty tissue on the first side; and
the second rib that intersects with the outflow tube segment and extends upwardly and rearwardly around the alar fibrofatty tissue on a second side of the nose and terminates at a second upper region on the second side of the mask to cooperate with the outflow tube segment thereby distributing load away from the alar fibrofatty tissue on the second side to a second region of the patient's face and/or nose above the alar fibrofatty tissue on the second side.

27. The capnography mask according to claim 23, further comprising:
a capnography port formed by a third tube segment that extends outwardly of the cover portion on the second side of the cover portion, the capnography port being fluidly connected to the breathing cavity for delivering a sample of the expired gas to a capnography machine.

28. A capnography system comprising:
the capnography mask according to claim 23, in which the inflow port and the outflow port are connected to a breathing circuit with tubing, and the capnography port is connected to a capnography machine with tubing.

29. The capnography system according to claim 28, wherein the breathing circuit includes a gas supply regulator that supplies the inflow gas to the inflow port via inflow tubing, and a vacuum source pulls the expired gas through the outflow port, and the capnography machine includes a separate vacuum source that pulls the sample of the expired gas through the capnography port.
